(19) United States
(12) Patent Application Publication
(10) Pub. No.: US 2022/0325322 A1
(43) Pub. Date: Oct. 13, 2022

(54) SEQUENCE SPECIFIC NUCLEIC ACID ENRICHMENT METHODS AND USES THEREOF

(71) Applicants: Vladimir I. SLEPNEV, San Jose, CA (US); Manasi RAJE, San Jose, CA (US); Nidhi GUPTA, San Jose, CA (US); Katie BOND, San Jose, CA (US); Frederick Edward TURNER, San Jose, CA (US); SHIELD DIAGNOSTICS CORP., San Jose, CA (US)

(72) Inventors: Vladimir I. SLEPNEV, Diamond Bar, CA (US); Manasi RAJE, San Dimas, CA (US); Nidhi GUPTA, San Dimas, CA (US); Katie BOND, San Dimas, CA (US); Frederick Edward TURNER, San Dimas, CA (US)

(21) Appl. No.: 17/631,852

(22) PCT Filed: Jul. 31, 2020
(86) PCT No.: PCT/US2020/044666

§ 371 Date: Jan. 31, 2022

Related U.S. Application Data
(60) Provisional application No. 62/882,420, filed on Aug. 2, 2019.

(51) Int. Cl. C12Q 1/689

(52) U.S. Cl. CPC C12Q 1/689; C12Q 1/6851

(57) ABSTRACT

The present disclosure, in some aspects, is directed to methods of nucleic acid enrichment using high affinity capture probes comprising one or more nucleotide analogs and uses thereof. For example, in another aspect, provided herein are methods of enriching a target nucleic acid if present in a sample. In another aspect, provided herein are methods of detecting if a target nucleic acid is in a sample. In another aspect, the present disclosure is directed to uses, kits, and compositions of the methods described herein.

### CROSS-REFERENCE TO RELATED APPLICATIONS

This application claims the priority benefit of U.S. Provisional Patent Application No. 62/882,420, filed on Aug. 2, 2019, the disclosure of which is hereby incorporated herein by reference in its entirety.

### TECHNICAL FIELD

The present disclosure, in some aspects, is directed to methods of nucleic acid enrichment. In other aspects, the present disclosure is directed to uses, kits, and compositions of the methods described herein.

### BACKGROUND

Methods that enable use of high affinity nucleic acid capture probes are desirable in the field of nucleic acid enrichment. While high affinity nucleic acid capture probes are known in the art, e.g., LNA capture probes, use of high affinity nucleic acid capture probes is limited by a lack of compatible elution techniques, such as elution techniques that have the ability to elute bound target nucleic acids, are compatible with downstream analyzes, and preserve the integrity of captured target nucleic acids. For example, it has been demonstrated that capture probes comprising all LNA sequences could not be used for target nucleic acid enrichment because it was not possible to elute the captured target nucleic acids using conventional methods, such as low ionic strength solutions and heat (Nucleic Acids Research, 2004, Vol. 32, No. 7 e64). Such limitations prevent full realization of high affinity captures probes for the enrichment of target nucleic acids.

All references cited herein, including patent applications and publications, are incorporated by reference in their entirety.

### BRIEF SUMMARY

In some aspects, the present disclosure provides a method of enriching a target nucleic acid, the method comprising: (a) contacting the sample with a capture probe under a condition that allows for formation of a complex comprising the capture probe and the target nucleic acid, wherein the capture probe comprises one or more nucleotide analogs; and (b) eluting the target nucleic acid from the capture probe using an elution buffer at a pH sufficient to disrupt the interaction between the target nucleic acid and the capture probe, thereby enriching the target nucleic acid if present in the sample.

In some embodiments, the capture probe comprising nucleotide analogs has increased binding affinity to the target nucleic acid, as compared to a capture probe without one or more of the nucleotide analogs.

In some embodiments, at least about 40% of the nucleotides of the capture probe are nucleotide analogs.

In some embodiments, the one or more nucleotide analogs are selected from any of a locked nucleic acid (LNA), peptide nucleic acid (PNA), a xeno nucleic acid (XNA), a glycol nucleic acid (GNA), a threose nucleic acid (TNA), a morpholino, a bridged nucleic acid (BNA), an O-methyl substituted RNA, a nucleotide with a modified sugar, base group, or backbone, or any combination thereof. In some embodiments, each of the one or more nucleotide analogs is a LNA.

In some embodiments, at least a portion of the capture probe is at least about 70% complementary to at least a portion of the target nucleic acid.

In some embodiments, the elution buffer has a pH of about 11 or greater. In some embodiments, the elution buffer has a pH of about 3 or less.

In some embodiments, the target nucleic acid is a RNA. In some embodiments, the target nucleic acid is a DNA.

In some embodiments, the capture probe is attached to a solid surface. In some embodiments, the capture probe is covalently attached to a solid surface.

In some embodiments, the methods described herein further comprise neutralizing the pH of the elution buffer after eluting the target nucleic acid.

In some embodiments, the methods described herein further comprise obtaining the sample. In some embodiments, the sample comprises a cell or cellular material.

In some embodiments, the methods described herein further comprise subjecting the sample to a lysing buffer comprising a chaotropic agent.

In some embodiments, the methods described herein further comprise detecting if the target nucleic acid is present. In some embodiments, the detecting the presence of the target nucleic acid is performed after eluting the target nucleic acid from the capture probe.

In another aspect, the present disclosure provides a method of detecting if a target nucleic acid is in a sample, the method comprising: (a) contacting the sample with a capture probe under a condition that allows for formation of a complex comprising the capture probe and the target nucleic acid, wherein the capture probe comprises one or more LNA nucleotides; (b) eluting the target nucleic acid from the capture probe using an elution buffer at a pH sufficient to disrupt the interaction between the target nucleic acid and the capture probe; (c) neutralizing the pH of the elution buffer after the eluting step; and (d) detecting if the target nucleic acid is present. In some embodiments, the detecting step comprises amplification using a DNA or RNA polymerase enzyme and subsequent detection of products of the amplification reaction.

These and other aspects and advantages of the present disclosure will become apparent from the subsequent detailed description and the appended claims. It is to be understood that one, some, or all of the properties of the various embodiments described herein may be combined to form other embodiments of the present disclosure.

### BRIEF DESCRIPTION OF THE DRAWINGS

FIG. 1 shows qRT-PCR relative fluorescence units (RFU) per cycle.

FIG. 2 shows qRT-PCR relative fluorescence units (RFU) per cycle.

FIG. 3 shows qRT-PCR relative fluorescence units (RFU) per cycle.

FIG. 4 shows qRT-PCR relative fluorescence units (RFU) per cycle.

FIG. 5 shows qRT-PCR relative fluorescence units (RFU) per cycle.

FIG. 6 shows qRT-PCR relative fluorescence units (RFU) per cycle.

### DETAILED DESCRIPTION

The present disclosure is based, in part, on the unexpected finding that aqueous solutions at extreme pH conditions (e.g., about 3 or less, or about 11 or greater) can be used to elute target nucleic acids bound to high affinity capture probes comprising nucleotide analogs without significantly degrading the captured and eluted target nucleic acids. The eluted material can then be easily neutralized without significant dilution, enabling downstream amplification reactions and detection. This finding is based on the discovery that interactions of capture probes comprising nucleotide analogs, e.g., LNA, with DNA or RNA can be disrupted by extreme pH conditions. In addition, surprisingly, we observed efficient elution of amplifiable RNA under strong alkaline conditions, a condition that is typically used to degrade RNA.

Thus, this disclosure, in some aspects, is directed to methods of nucleic acid enrichment. In other aspects, the present disclosure is directed to uses, kits, and compositions of the methods described herein. For example, in some embodiments, the disclosure encompasses methods and compositions for extracting nucleic acids from cells in complex biological samples or specimens by hybridizing target nucleic acids to oligonucleotide capture probes comprising nucleotide analogs and subsequently releasing bound target nucleic acid using an elution buffer having a desired pH. The disclosure further provides techniques for amplification and detection of target nucleic acids, techniques that can be particularly useful in diagnostic assay development.

Nucleotide analogs, such as DNA analogs, that possess higher affinity to form duplexes with complementary DNA or RNA, while at the same time obeying the Watson-Crick base-pairing rules are especially useful for sequence-specific enrichment. Such nucleotide analogs are exemplified by LNA nucleotides, bi-cyclic compounds structurally very similar to RNA-monomers (as described in U.S. Pat. No. 6,303,315, which is hereby incorporated by reference in its entirety). LNA oligonucleotides are synthesized by standard phosphoramidite chemistry and have high affinity toward complementary DNA or RNA, significantly exceeding the affinity of natural nucleotides (Tetrahedron, 54, 3607-3630 (1998)). The affinity of LNA/DNA and LNA/RNA heteroduplexes is sufficiently high to allow efficient hybridization to occur even in the presence of highly chaotropic agents such as guanidine thiocyanate (GnSCN) (Nucleic Acids Research, 2004, Vol. 32, No. 7 e64). It is especially advantageous to conduct hybridization to LNA probes under such conditions since it helps to disrupt interactions with nucleic acids and proteins originating from the sample, and to remove secondary and tertiary structures in nucleic acids. At the same time, high affinity interaction enabled by LNA oligonucleotides prevents efficient recovery of bound nucleic acids. For example, oligonucleotides comprising all LNA sequences could not be used because elution of bound nucleic acids using conventional methods, such as low ionic strength solutions and heat, is not possible (Nucleic Acids Research, 2004, Vol. 32, No. 7 e64). This limitation prevents full realization of binding affinity offered by LNA and requires incorporation of DNA nucleotides in probes to enable practical elution of bound nucleic acid material. Nucleic acid base pairing could be further disrupted by organic solvents (such as formamide), however such solvents will also impair downstream reactions using DNA or RNA modifying enzymes.

Since the pioneering work by Saton and Inue (Biochem J. 1969; 114(2):271-7) that demonstrated complete RNA hydrolysis to mono-, di-, and trinucleotides within minutes at slightly elevated temperature (37° C.), RNA hydrolysis using alkali has become a standard procedure incorporated in a variety of molecular techniques. We have demonstrated that elution and neutralization within 1-5 minutes yielded RNA of significant length that could be amplified in Reverse transcription-PCR reaction.

Also contemplated herein are the components and kits of the methods disclosed herein.

It will also be understood by those of ordinary skill in the art that changes in the form and details of the implementations described herein may be made without departing from the scope of this disclosure. In addition, although various advantages, aspects, and objects have been described with reference to various implementations, the scope of this disclosure should not be limited by reference to such advantages, aspects, and objects.

### Definitions

The terms “comprising,” “having,” “containing,” and “including,” and other similar forms, and grammatical equivalents thereof, as used herein, are intended to be equivalent in meaning and to be open ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of.”

Where a range of values is provided, it is understood that each intervening value, to the tenth of the unit of the lower limit, unless the context clearly dictate otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

Reference to “about” a value or parameter herein includes (and describes) variations that are directed to that value or parameter per se. For example, description referring to “about X” includes description of “X.”

As used herein, including in the appended claims, the singular forms “a,” “or,” and “the” include plural referents unless the context clearly dictates otherwise.

Those skilled in the art will recognize that several embodiments are possible within the scope and spirit of the present disclosure. The following description illustrates the disclosure and, of course, should not be construed in any way as limiting the scope of the inventions described herein.

In some embodiments, provided herein are methods of method of enriching a target nucleic acid in a sample, the method comprising contacting the sample with a capture probe under a condition that allows for formation of a complex comprising the capture probe and the target nucleic acid, wherein the capture probe comprises one or more nucleotide analogs.

In some embodiments, provided herein are methods of enriching a target nucleic acid, if present in a sample, the method comprising: (a) contacting the sample with a capture probe under a condition that allows for formation of a complex comprising the capture probe and the target nucleic acid, if present in the sample, wherein the capture probe comprises one or more nucleotide analogs; and (b) eluting the target nucleic acid from the capture probe, if the complex is formed, using an elution buffer at a pH sufficient to disrupt the interaction between the target nucleic acid and the capture probe, thereby enriching the target nucleic acid if present in the sample.

In some embodiments, the capture probe comprising nucleotide analogs has increased binding affinity to the target nucleic acid, as compared to a capture probe without one or more of the nucleotide analogs. In some embodiments, at least about 40%, such as at least about any of 45%, 50%, 55%, 60%, 65%, 70%, 75%, 80%, 85%, 90%, or 95%, of the nucleotides of the capture probe are nucleotide analogs. In some embodiments, all of the nucleotides of the capture probe are nucleotide analogs. In some embodiments, at least about 40%, such as at least about any of 45%, 50%, 55%, 60%, 65%, 70%, 75%, 80%, 85%, 90%, or 95%, of the nucleotides of the capture probe are LNAs. In some embodiments, all of the nucleotides of the capture probe are LNAs.

In some embodiments, the one or more nucleotide analogs are selected from any of a locked nucleic acid (LNA), peptide nucleic acid (PNA), a xeno nucleic acid (XNA), a glycol nucleic acid (GNA), a threose nucleic acid (TNA), a morpholino, a bridged nucleic acid (BNA), an O-methyl substituted RNA, a nucleotide with a modified sugar, base group, or backbone, or any combination thereof. In some embodiments, the one or more nucleotide analogs is an LNA.

In some embodiments, at least a portion of the capture probe, such as at least about 5 nucleotides, is at least about 70% complementary to at least a portion of the target nucleic acid.

In some embodiments, the capture probe comprises a hybridizing portion, wherein the hybridizing portion of the capture probes comprises a nucleic acid sequence, such as two or more consecutive nucleotides, capable of hybridizing to a target nucleic acid or a portion thereof. In some embodiments, the hybridizing portion of a capture probe comprises about 5 nucleotides to about 50 nucleotides, such as about 5 nucleotides to about 25 nucleotides, about 10 nucleotides to about 20 nucleotides, or about 20 nucleotides to about 40 nucleotides. In some embodiments, the hybridizing portion of a capture probe comprises at least about 5 nucleotides, such as at least about any of 10 nucleotides, 15 nucleotides, 20 nucleotides, 25 nucleotides, 30 nucleotides, 35 nucleotides, 40 nucleotides, 45 nucleotides, or 45 nucleotides. In some embodiments, the hybridizing portion of a capture probe comprises less than about 45 nucleotides, such as less than about any of 40 nucleotides, 35 nucleotides, 30 nucleotides, 25 nucleotides, 20 nucleotides, 15 nucleotides, or 10 nucleotides. In some embodiments, the hybridizing portion of a capture probe comprises any of 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, 30 nucleotides, 31 nucleotides, 32 nucleotides, 33 nucleotides, 34 nucleotides, 35 nucleotides, 36 nucleotides, 37 nucleotides, 38 nucleotides, 39 nucleotides, 40 nucleotides, 41 nucleotides, 42 nucleotides, 43 nucleotides, 44 nucleotides, 45 nucleotides, 46 nucleotides, 47 nucleotides, 48 nucleotides, 49 nucleotides, or 50 nucleotides.

In some embodiments, the hybridizing portion of a capture probe comprises the one or more nucleotide analogs. In some embodiments, the nucleotide analog is an analog that obeys Watson-Crick base pairing. In some embodiments, the nucleotide analog hybridizes to a complementary nucleotide, such as a naturally occurring nucleotide, with stronger affinity than a naturally occurring complementary nucleotide. In some embodiments, the nucleotide analog is selected from the group consisting of a locked nucleic acid (LNA), a peptide nucleic acid (PNA), a xeno nucleic acid (XNA), a glycol nucleic acid (GNA), a threose nucleic acid (TNA), a morpholino, a bridged nucleic acid (BNA), an O-methyl substituted RNA, and a nucleotide with one or more sugar, base group, or backbone modifications, or a combination thereof.

In some embodiments, the nucleotides of a hybridizing portion of a capture probe comprises about 40% to about 100% nucleotide analogs, such as about 40% to about 80% nucleotide analogs, about 60% to about 90% nucleotide analogs, about 75% to about 100% nucleotide analogs, or about 90% to about 100% nucleotide analogs. In some embodiments, the nucleotides of a hybridizing portion of a capture probe comprises at least about 40%, such as at least about any of 45%, 50%, 55%, 60%, 65%, 70%, 75%, 80%, 85%, 90%, 95%, 96%, 97%, 98%, 99%, or 100%, nucleotide analogs. In some embodiments, the nucleotides of a hybridizing portion of a capture probe comprise about any of 80% nucleotide analogs, 81% nucleotide analogs, 82% nucleotide analogs, 83% nucleotide analogs, 84% nucleotide analogs, 85% nucleotide analogs, 86% nucleotide analogs, 87% nucleotide analogs, 88% nucleotide analogs, 89% nucleotide analogs, 90% nucleotide analogs, 91% nucleotide analogs, 92% nucleotide analogs, 93% nucleotide analogs, 94% nucleotide analogs, 95% nucleotide analogs, 96% nucleotide analogs, 97% nucleotide analogs, 98% nucleotide analogs, 99% nucleotide analogs, or 100% nucleotide analogs. In some embodiments the nucleotide analog is an LNA. In some embodiments, the nucleotide analog is selected from the group consisting of a peptide nucleic acid (PNA), a xeno nucleic acid (XNA), a glycol nucleic acid (GNA), a threose nucleic acid (TNA), a morpholino, a bridged nucleic acid (BNA), an O-methyl substituted RNA, and a nucleotide with one or more sugar, base group, or backbone modifications, or a combination thereof.

In some embodiments, the hybridizing portion of a capture probe is designed to have a desired affinity for a target nucleic acid or a portion thereof. The affinity of the a hybridizing portion for a target nucleic acid or a portion thereof may be tuned based on, e.g., length (e.g., number of nucleotides), number of consecutive nucleotides, degree of complementarity to a target sequence, inclusion of modified nucleotides, inclusion of a space and/or a linker, such as spacer or linker bases, inclusion of mismatched nucleotides, and amount of modified high affinity nucleotides. In some embodiments, the hybridizing portion of a capture probe is designed to have a desired affinity for a target nucleic acid, or a portion thereof, in a specific condition (such as in a denaturing condition, e.g., a solution of 4M guanidinium salts, and/or an elevated temperature, e.g., about 30° C. to about 60° C.). In some embodiments, the capture probe comprises more than one hybridizing portion, such as any of 2, 3, 4, or 5 hybridizing portions.

In some embodiments, the hybridizing portion of a capture probe is complementary to a target nucleic acid or a portion thereof. In some embodiments, the hybridizing portion of a capture probe has at least about 60%, such as at least about any of 65%, 70%, 75%, 80%, 85%, 90%, 95%, 96%, 97%, 98%, or 99%, sequence complementarity to a target nucleic acid or a portion thereof.

In some embodiments, the desired affinity for a target nucleic acid or a portion thereof is based on a melting temperature threshold affinity. The threshold affinity for oligonucleotide hybridization may be estimated using Tₘ (melting temperature) calculation. It is noted that Tₘ calculation is highly dependent on, e.g., ionic conditions, on the nature of cations, presence of denaturants, and the extent of incorporation of high affinity bases (e.g., LNA) into the capture probe. Thus, a capture probe designed to have a desired affinity for a target nucleic acid or a portion thereof will have a desired affinity for a target nucleic acid or a portion thereof in a relevant condition. In some embodiments, empirically, the capture probe binding to the target nucleic acid under conditions denaturing conditions (e.g., 4 M guanidinium salt) can be measured and used as a threshold affinity.

In some embodiments, the capture probe comprising one or more nucleotide analogs is a polynucleotide or oligonucleotide containing one or more LNA monomers and a variable number of naturally occurring nucleotides or their analogues, such as 7-deazaguanosine or inosine, and is sufficiently complementary to hybridize with the target nucleic acid such that stable and specific binding occurs to form a complex between the target and the complementary nucleic acid under the hybridization conditions. In some embodiments, the capture probe sequence need not reflect the exact sequence of the target nucleic acid. For example, a non-complementary nucleotide fragment may be attached to a complementary nucleotide fragment or alternatively, non-complementary bases or longer sequences can be interspersed into the complementary nucleic acid, provided that the complementary nucleic acid sequence has sufficient complementarity with the sequence of the target nucleic acid to hybridize therewith, forming a hybridization complex and further is capable of immobilizing the target nucleic acid to a solid support as will be described in further detail below.

In some embodiments, the capture probe comprises a linker and/or a spacer moiety.

In some embodiments, the capture probe is attached to a partner of a binding group (e.g., biotin/avidin, fluorescein or carboxyfluorescein/albumin, magnetic micro-particle, a component of a click chemistry group), such as via the linker. In some embodiments, the capture probe is associated with a capture medium, e.g., a solid phase or particle, such as via the linker. In some embodiments, the capture probe is bound, such as covalently bound, to a capture medium, e.g., a solid phase or particle, such as via the linker. In some embodiments, the capture probe is associated with or bound to a capture medium via the binding group.

In some embodiments, the linker comprises one or more nucleotides, or analogs or derivatives thereof, such as a nucleotide that will not hybridize to a target nucleic acid or a portion thereof when the hybridizing portion of the capture probe is hybridized thereto. In some embodiments, the linker comprises a polymer, such as a linear polymer. In some embodiments, the polymer is based on a nucleic acid backbone structure. In some embodiments, the linker comprises phosphoramidite, such as phosphoramidite C3, phosphoramidite 9, phosphoramidite C12, or phosphoramidite 18. In some embodiments, the length of the linker is equal to the length of a linear single-stranded nucleic acid that is about 1 nucleotide to about 100 nucleotides, such as about 1 nucleotide to about 50 nucleotides, about 5 nucleotides to about 50 nucleotides, or about 5 nucleotides to about 100 nucleotides.

In some embodiments, the spacer moiety separates, such as sits between on a linear nucleic acid, two parts of a hybridizing portion of a capture probe. In some embodiments, the spacer moiety separates a hybridizing portion and a linker of a capture probe. In some embodiments, the spacer moiety comprises non-hybridizing nucleotides, e.g., nucleotides selects based on the target nucleic acid sequence. In some embodiments, the length of the spacer moiety is equal to the length of a linear single-stranded nucleic acid that is about 1 nucleotide to about 50 nucleotides, such as about 5 nucleotides to about 50 nucleotides.

In some embodiments, the affinity of a capture probe can be tuned, such as further tuned, by incorporating one or more spacers and/or linkers that do not hybridize to a target nucleic acid, For example, in some embodiments, the affinity of a capture probe is tuned using one or more spacer moieties positioned in the capture probe to separate hybridizing portions of the capture probe. In some embodiments, inclusion of the spacer moiety reduces the total number of complementary nucleotides and nucleotide analogs present in the capture probe for a specific target nucleic acid. In some embodiments, inclusion of the spacer moiety enables specificity of complementarity over a span of the target nucleic acid, without having a capture probe that is complementary to the corresponding entire stretch of the target nucleic acid. In some embodiments, for a capture probe comprising more than one hybridizing portions separated by a spacer, the segmented hybridizing portions contain complementary sequences to the target nucleic acid, the hybridizing portions comprising one or more LNA and/or DNA nucleotide analogs. In some embodiments, for a capture probe comprising more than one hybridizing portions separated by a spacer, the hybridizing portions comprise a sequence of about 6 to about 20 nucleotides (such as LNA), and wherein the hybridizing portions are separated by a spacer and/or a linker with a length that is equal to the length of a linear single-stranded nucleic acid that is about 5 nucleotides to about 20 nucleotides. In some embodiments, the capture probe comprises two hybridizing portions separated by a spacer, wherein each of the hybridizing portions comprise a sequence of about 6 to about 20 nucleotides (such as LNA), and wherein the hybridizing portions are separated by a spacer with a length that is equal to the length of a linear single-stranded nucleic acid that is about 5 nucleotides to about 20 nucleotides.

In some embodiments, the capture probe is associated with a surface of a capture medium. In some embodiments, the capture probe is attached to a surface of a capture medium via a linker, wherein the length of the linker is equal to the length of a linear single-stranded nucleic acid that is about 5 nucleotides to about 100 nucleotides. In some embodiments, the capture probe is attached (such as conjugated) to a surface of a capture medium. In some embodiments, the capture probe comprises one or more branching nucleotides, wherein multiple target binding sequences are linked to the single attachment site at the surface of the capture medium.

In some embodiments, the density of the capture probes on the capture medium is about 5 nanomole of capture probe per mL of capture medium to about 250 nanomole of capture probe per mL of capture medium, such as about 10 nanomole of capture probe per mL of capture medium to about 100 nanomole of capture probe per mL of capture medium. In some embodiments, the density of the capture probes on the capture medium is at least about 10 nanomole per mL of capture medium, such as at least about any of 20, such as at least about any of 30, 40, 50, 60, 70, 80, 90, 100, 125, 150, 175, 200, 225, or 250, nanomole per mL of capture medium.

In some embodiments, the capture medium has a single species of capture probes (e.g., capture probes comprising identical hybridizing portions) associated therewith. In some embodiments, the capture medium has two or more species of capture probes (e.g., capture probes capable of hybridizing different target nucleic acids or two species of capture probes capable of hybridizing the same target nucleic acid) associated therewith.

In some embodiments, the capture medium is a matrix. In some embodiments, the capture medium is resistant to shrinking and/or swelling, such as during exposure to buffers of various ion strength and/or pH. In some embodiments, the capture medium is a plurality of particles, such as beads. In some embodiments, the capture medium comprises a plurality of magnetic beads, such as polymer-coated magnetic beads. In some embodiments, the largest dimension of each of the particles is about 5 μm to about 300 μm, such as about 25 μm to about 100 μm, about 50 μm to about 100 μm, about 50 μm to about 90 μm, about 50 μm to about 250 μm, or about 100 μm to about 250 μm. In some embodiments, the largest dimension of each of the particles is at least about 5 μm, such as at least about any of 10 μm, 15 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 110 μm, 120 μm, 130 μm, 140 μm, 150 μm, 160 μm, 170 μm, 180 μm, 190 μm, 200 μm, 225 μm, 250 μm, 275 μm, or 300 μm. In some embodiments, the largest dimension of each of the particles is less than about 300, such as less than about any of 275 μm, 250 μm, 225 μm, 200 μm, 190 μm, 180 μm, 170 μm, 160 μm, 150 μm, 140 μm, 130 μm, 120 μm, 110 μm, 100 μm, 95 μm, 90 μm, 85 μm, 80 μm, 75 μm, 70 μm, 65 μm, 60 μm, 55 μm, 50 μm, 45 μm, 40 μm, 35 μm, 30 μm, 25 μm, 20 μm, 15 μm, 10 μm, or 5 μm. In some embodiments, the largest dimension of each of the particles is about any of 5 μm, 10 μm, 15 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 110 μm, 120 μm, 130 μm, 140 μm, 150 μm, 160 μm, 170 μm, 180 μm, 190 μm, 200 μm, 225 μm, 250 μm, 275 μm, or 300 μm.

In some embodiments, the capture medium is composed of a material comprising a polyacrylic or polymethacrylic polymer. In some embodiments, the capture medium is composed of a material comprising a polystyrene or crosslinked polystyrene.

In some embodiments, the capture medium is a 5′-dimethoxytrityl-adenosine-2′,3′-diacetate-N-linked-polymeric support. In some embodiments, the capture medium is an Oligo-Affinity Support (PS) (Glen Research, Sterling, Va.).

In some embodiments, the elution buffer has a pH of about 10 or greater, such as about 10.1 or greater, about 10.2 or greater, about 10.3 or greater, about 10.4 or greater, about 10.5 or greater, about 10.6 or greater, about 10.7 or greater, about 10.8 or greater, about 10.9 or greater, about 11 or greater, about 11.1 or greater, about 11.2 or greater, about 11.3 or greater, about 11.4 or greater, about 11.5 or greater, about 11.6 or greater, about 11.7 or greater, about 11.8 or greater, about 11.9 or greater, about 12 or greater, about 12.1 or greater, about 12.2 or greater, about 12.3 or greater, about 12.4 or greater, about 12.5 or greater, about 12.6 or greater, about 12.7 or greater, about 12.8 or greater, about 12.9 or greater, about 13 or greater, about 13.1 or greater, about 13.2 or greater, about 13.3 or greater, about 13.4 or greater, about 13.5 or greater, about 13.6 or greater, about 13.7 or greater, about 13.8 or greater, about 13.9 or greater, or about 14 or greater. In some embodiments, the elution buffer has a pH of about any of 10, 10.1, 10.2, 10.3, 10.4, 10.5, 10.6, 10.7, 10.8, 10.9, 11, 11.1, 11.2, 11.3, 11.4, 11.5, 11.6, 11.7, 11.8, 11.9, 12, 12.1, 12.2, 12.3, 12.4, 12.5, 12.6, 12.7, 12.8, 12.9, 13, 13.1, 13.2, 13.3, 13.4, 13.5, 13.6, 13.7, 13.8, 13.9, or 14. In some embodiments, the elution buffer comprises a hydroxide, such as any one or more of sodium hydroxide, potassium hydroxide, and lithium hydroxide.

In some embodiments, the elution buffer has a pH of about 4 or less, such as about 3.9 or less, about 3.8 or less, about 3.7 or less, about 3.6 or less, about 3.5 or less, about 3.4 or less, about 3.3 or less, about 3.2 or less, about 3.1 or less, about 3 or less, about 2.9 or less, about 2.8 or less, about 2.7 or less, about 2.6 or less, about 2.5 or less, about 2.4 or less, about 2.3 or less, about 2.2 or less, about 2.1 or less, about 2 or less, about 1.9 or less, about 1.8 or less, about 1.7 or less, about 1.6 or less, about 1.5 or less, about 1.4 or less, about 1.3 or less, about 1.2 or less, about 1.1 or less, about 1 or less, about 0.9 or less, about 0.8 or less, about 0.7 or less, about 0.6 or less, about 0.5 or less, about 0.4 or less, about 0.3 or less, about 0.2 or less, or about 0.1 or less. In some embodiments, the elution buffer has a pH of about any of 4, 3.9, 3.8, 3.7, 3.6, 3.5, 3.4, 3.3, 3.2, 3.1, 3, 2.9, 2.8, 2.7, 2.6, 2.5, 2.4, 2.3, 2.2, 2.1, 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2, or 0.1. In some embodiments, the elution buffer comprises an acid, such as any one or more of hydrochloric acid, sulfuric acid, phosphoric acid, formic acid, and trifluoracetic acid.

In some embodiments, the volume of elution buffer used in eluting the target nucleic acid from the capture probe is between about 500 μL to about 20 μL. In some embodiments, the volume of elution buffer used in eluting the target nucleic acid from the capture probe is less than about 500 μL, such as less than about any of 450 μL, 400 μL, 350 μL, 300 μL, 250 μL, 200 μL, 150 μL, 100 μL, 75 μL, 50 μL, or 25 μL.

In some embodiments, the method comprises eluting the target nucleic acid from the capture probe using an elution buffer at an extreme pH, such as an elution buffer at a pH of about <3 or >11, by exposing the capture probe hybridized to a target nucleic acid to the elution buffer for less than about 10 minutes, such as less than about any of 9 minutes, 8, minutes, 7 minutes, 6 minutes, 5 minutes, 4 minutes, 3 minutes, 2 minutes, 1 minute, or 30 seconds. In some embodiments, the capture probe hybridized to a target nucleic acid is subjected to the elution buffer at a temperature of about 20° C. to about 100° C. In some embodiments, the capture probe hybridized to a target nucleic acid is subjected to the elution buffer at a temperature of at least about 20° C., such as at least about any of 25° C., 30° C., 35° C., 40° C., 45° C., 50° C., 55° C., 60° C., 65° C., 70° C., 75° C., 80° C., 85° C., 90° C., 95° C., or 100° C.

In some embodiments, the target nucleic acid is a deoxyribonucleic acid (DNA). In some embodiments, the target nucleic acid is a ribonucleic acid (RNA), including a messenger RNA (mRNA), a ribosomal ribonucleic acid (rRNA), a transfer RNA, (tRNA), a small nuclear (snRNA), a telomerase associated RNA, or a ribozyme associated RNA.

In some embodiments, the nucleic acid sample of interest will be one which is suspected of containing a particular target nucleic acid, such as a particular gene, gene segment or RNA. In some embodiments, of particular interest is the detection of particular mRNAs which may be of eukaryotic, prokaryotic, archaeal or viral origin. In some embodiments, the invention may assist in the diagnosis of various infectious diseases by assaying for particular sequences known to be associated with a particular microorganism.

In some embodiments, the target nucleic acid may be provided in a complex biological mixture of nucleic acid (RNA, DNA and/or rRNA) and non-nucleic acid. In some embodiments, the target nucleic acid is RNA molecules. In some embodiments, the target nucleic acid is rRNAs, such as the 16S or 23S rRNA, such as described in U.S. Ser. No. 08/142,106, which is incorporated by reference herein in its entirety.

In some embodiments, if target nucleic acids of choice are double stranded or otherwise have significant secondary and tertiary structure, they may need to be heated prior to hybridization. In some embodiments, in this case, heating may occur prior to or after the introduction of nucleic acids into the hybridization medium containing the capturing probe. In some embodiments, it may also be desirable in some cases to extract the nucleic acids from the complex biological samples prior to the hybridization assay to reduce background interference by any methods known in the art.

In some embodiments, the hybridization and extraction methods of the present invention may be applied to a complex biological mixture of nucleic acid (RNA and/or DNA) and non-nucleic acid. In some embodiments, such a complex biological mixture includes a wide range of eukaryotic and prokaryotic cells, including protoplasts; or other biological materials which may harbor target nucleic acids. In some embodiments, the methods are thus applicable to tissue culture cells, human cells (e.g., blood, serum, plasma, reticulocytes, lymphocytes, urine, bone marrow tissue, cerebrospinal fluid or any product prepared from blood or lymph) or any type of tissue biopsy homogenized in lysis buffer), plant cells or other cells sensitive to osmotic shock and cells of bacteria, yeasts, viruses, protozoa, fungi and other microbial cells and the like.

In some embodiments, the assay and isolation procedures of the present invention are useful, for instance, for detecting non-pathogenic or pathogenic micro-organisms of interest. In some embodiments, by detecting specific hybridization between nucleotide probes of a known source and nucleic acids resident in the biological sample, the presence of the micro-organisms may be established.

Solutions containing high concentrations of guanidine, guanidinium thiocyanate or certain other chaotropic agents and detergents are capable of effectively lysing prokaryotic and eukaryotic cells while simultaneously allowing specific hybridization of LNA probes to the released endogenous nucleic acid. In some embodiments, the solutions need not contain any other component other than common buffers and detergents to promote lysis and solubilization of cells and nucleic acid hybridization.

In some embodiments, the capture probe comprising one or more nucleotide analogs with higher binding affinity, for example LNA, substantially complementary to the target nucleic acid are used in the hybridization process. In some embodiments, the capture probe comprising one or more nucleotide analogs is a polynucleotide or oligonucleotide containing one or more LNA monomers and a variable number of naturally occurring nucleotides or their analogues, such as 7-deazaguanosine or inosine, and is sufficiently complementary to hybridize with the target nucleic acid such that stable and specific binding occurs to form a complex between the target and the complementary nucleic acid under the hybridization conditions. In some embodiments, the capture probe sequence need not reflect the exact sequence of the target nucleic acid. For example, a non-complementary nucleotide fragment may be attached to a complementary nucleotide fragment or alternatively, non-complementary bases or longer sequences can be interspersed into the complementary nucleic acid, provided that the complementary nucleic acid sequence has sufficient complementarity with the sequence of the target nucleic acid to hybridize therewith, forming a hybridization complex and further is capable of immobilizing the target nucleic acid to a solid support as will be described in further detail below.

In some embodiments, the capture probe comprises a linker or a spacer moiety. In some embodiments, the capture probe is attached to a binding group (e.g. biotin, fluorescein, magnetic micro-particle etc.), such as via the linker or the spacer moiety. In some embodiments, the capture probe is bound, such as covalently bound, to a solid phase or particle.

In some embodiments, the described invention is possible with other nucleotide analogs with higher binding affinity than DNA, for example PNA (peptide nucleic acid), XNA (xeno nucleic acid), GNA (glycol nucleic acid), TNA (threose nucleic acid), morpholinos, BNA (bridged nucleic acid), O-methyl substituted RNA or nucleotides with a modified sugar, base group, backbone or some combination of these that serves to increase the binding affinity for DNA and RNA.

In some embodiments, the target nucleic acid eluted using an elution buffer at an extreme pH, such as an elution buffer at a pH of about <3 or >11, is subsequently neutralized following elution. In some embodiments, the target nucleic acid is subject to extreme pH for less than about 10 minutes, such as less than about any of 9 minutes, 8, minutes, 7 minutes, 6 minutes, 5 minutes, 4 minutes, 3 minutes, 2 minutes, 1 minute, or 30 seconds.

In some embodiments, the methods described herein comprise further steps, such as any one or more of neutralizing the pH of the elution buffer after eluting the target nucleic acid, obtaining the sample, subjecting the sample to a lysing buffer comprising a chaotropic agent, and detecting if the target nucleic acid is present. In some embodiments, pH neutralization comprises admixing a buffer (such as a having a desired pH and at a desire quantity) with the elution buffer or a composition comprising the elution buffer after eluting the target nucleic acid from the capture probe.

The disclosure provided herein is further illustrated by the following examples, which are not to be construed as limiting the disclosure in scope or spirit to the specific procedures described in them.

### EXAMPLES

### Example 1

The synthesis of LNA probes was carried out on solid support functionalized with a DMT-blocked monomer on an Expedite 8909 DNA Synthesizer (Biolytic Inc., Fremont, Calif.). The solid support used in this invention was Oligo Affinity Support which is a column packed with polystyrene beads. The stability of the linkage between polystyrene support and the first monomer under deprotection conditions is appreciated in this invention. The oligonucleotides were synthesized on the oligo affinity support and deprotected while conserving attachment of the probes to the support. As a result, the support can be used as a capture device going forward.

In other aspects of the invention it might be necessary that the probes be cleaved and re-attached to another preferred support for target capture and in such a case, controlled pore glass support can be used in the process of synthesis.

All DNA phosphoramidites, spacer phosphoramidite and LNA phosphoramidites with exception of dC LNA phosphoramidite were dissolved in anhydrous acetonitrile with 10% weight/volume activated molecular sieves to further dry the solution. dC LNA phosphoramidite was dissolved in 1:1 dichloromethane/acetonitrile with 20% weight/volume activated molecular sieves.

The final concentration of all LNA phosphoramidites is listed in Table 1. Preparation of chemicals was done at room temperature inside a fume-hood. The solutions were loaded on the Expedite synthesizer as soon as they were prepared.

TABLE 1
LNA phosphoramidites and spacer 18 concentration used
for synthesis of LNA probes.

| | LNA dA | LNA dC | LNA dG | LNA dT | Spacer 18 |
|---|---|---|---|---|---|
| Reagent | phosphoramidite | phosphoramidite | phosphoramidite | phosphoramidite | phosphoramidite |
| Concentration | 66.40 | 67.15 | 65.14 | 68.10 | 98.00 |
| (mM) | | | | | |

The synthesis was carried out using standard phosphoramidite chemistry. The Oligo-Affinity solid (OAS) support (5′-Dimethoxytrityl-Adenosine-2′,3′-diacetate-N-Linked-Polymeric Support, Glenn Research), carrying the first monomer was detritylated and then the second monomer of Spacer-18 activated by 1-H tetrazole in anhydrous acetonitrile was coupled.

Activation was also performed in different examples using 0.38 M dicyanoimidazole in acetonitrile. The unreacted hydroxyl groups were capped and the phosphite triester bond formed in the coupling reaction was oxidized using iodine, base and water. Subsequent monomers were added using the same protocol.

The capture probes were synthesized comprising multiple Spacer-18 monomers to make a longer linker followed by LNA phosphoramidites. Post synthesis, the solid support carrying the synthesized oligonucleotides was treated with 4 ml concentrated aqueous ammonia for 17 hours at 55° C. to deprotect the protected bases and backbone. After deprotection the solid support column was flushed with deionized water at room temperature until neutral pH was observed. The column was then dried with air by injecting air into the column multiple times. The column was then packed in a sealed bag and stored at 6-8 C until used for target capture.

LNA oligonucleotides of the following sequence were synthesized:

(SEQ ID NO: 1)
5′-(dT)15-(Spacer-18)5-OAS
(SEQ ID NO: 2)
5′-GGGCGGTGTGTACAAG-(Spacer 18)5-OAS

### Example 2: RNA Capture with Alkaline Elution

### Sample Preparation

Escherichia coli cells were grown in LB media (American Bioinnovations), then spun down and resuspended in 3 mL of 1× Phosphate Buffered Saline (Sigma-Aldrich). This was then pipetted into Tempus Blood RNA tubes (Thermo Fisher) containing 6 mL stabilizing solution comprising GnSCN. Triton X-100 (Sigma-Aldrich) was added to the solution for a final concentration of 1%. The prepared sample was mixed by inversion and stored for 30 minutes at room temperature prior to RNA capture.

### RNA Capture

1 mL of the prepared sample was flowed through the Oligo Affinity Support Column (˜60 μl volume) containing the LNA capture probe SEQ ID NO:2 for hybridization. Immediately following hybridization, 1 mL of Wash Buffer 1 (3 M guanidinium thiocyanate (Sigma-Aldrich), 10 mM Tris-HCl (pH 8.5, Rockland), 1% Triton X-100 (Sigma-Aldrich)) was flowed through the column, followed by 1 mL of Wash Buffer 2 (150 mM NaCl (Sigma-Aldrich), 100 mM Tris-HCl (pH 8.5 (Rockland)), 0.02% Triton X-100 (Sigma-Aldrich)) followed by 1 mL of Wash Buffer 3 (150 mM NaCl (Sigma-Aldrich), 50 mM Tris-HCl (pH 8.5) (Rockland)). After Wash Buffers 1, 2, and 3, the column was cleared by air.

400 μl of elution solution (0.1 M NaOH (Alfa Aesar)) was flowed through the column and collected in 10 fractions of 40 μl each. The fractions were neutralized with 0.5 M HCl (Merck). Tris-HCl (pH 8.0) (Thermo Fisher) was added to a final concentration of 50 mM. The elutions were stored at −80° C. for seven days prior to quality control testing.

### Quantitative Reverse Transcription PCR

The first 6 fractions of elution solution were reverse transcribed and amplified using specific primers and a probe for conserved bacterial 16S. qRT-PCR reactions (25 μl) were prepared by mixing 1× HawkZ05 Fast One-Step RT-PCR Master (Roche), 1.5 mM manganese acetate (Roche), 1 mM MgCl2 (Thermo Fisher), 4% DMSO (Thermo Fisher), 0.4 μM forward primer (IDT), 0.4 μM reverse primer (IDT), 0.2 μM probe (IDT) and eluted RNA as template (5 μl). The cycling protocol was performed as following: 5 minutes at 55° C., 10 minutes at 60° C., 15 minutes at 65° C., 3 minutes at 94° C., and 60 repetitions of the following cycle: 10 seconds at 94° C., 15 seconds at 60° C., and 3 seconds at 72° C. (plate readings captured at the 72° C. step of each cycle). The specific oligonucleotides used were as follows (Table 2):

TABLE 2
Primers and probes.

| Function | Sequence |
|---|---|
| Forward | 5′ GAT GGT GCA GGT GGT GCA /iSuper-dT/GG 3′ 3′ |
| primer | (SEQ ID NO: 3) |
| Reverse | 5′ GTG GGT TGC GCT CGT TGC GG 3′ 3′ (SEQ ID NO: 4) |
| primer | |
| qPCR probe | 5′ /56-FAM/G+TCG+TC+AGC+TCG+TG/Iowa Black FQ/ 3′ 3′ |
| | (SEQ ID NO: 5) |

### Results and Discussion

Escherichia coli was grown and suspended in Phosphate Buffered Saline then added to Tempus RNA tubes and combined with additional detergent. This solution was subject to the LNA 16S Oligo Affinity Support Column capture protocol as above. The quality of the captured RNA was subsequently assessed by qRT-PCR.

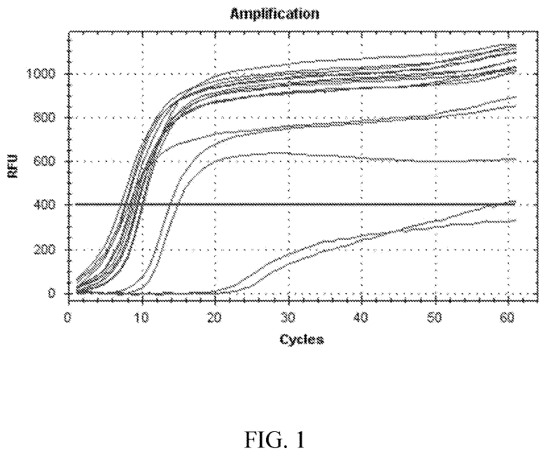

FIG. 1 and Table 3 show the results of qRT-PCR, in which 6 elutions were amplified in qRT-PCR reactions using specific primers and a probe for conserved bacterial 16S rRNA. All elutions show considerable amplification, with the most 16S rRNA detected in Elution 2. The results demonstrate the ability to selectively capture and elute amplifiable bacterial RNA in Alkaline buffer (pH>12) without substantial loss of full-length RNA, as would be expected in an alkaline solution.

TABLE 3
qRT-PCR average cycle threshold.

| Sample | Average Cycle Threshold |
|---|---|
| Negative Control | 58.53 |
| Positive Control | 14.32 |
| Elution 1 | 8.57 |
| Elution 2 | 7.65 |
| Elution 3 | 7.73 |
| Elution 4 | 9.11 |
| Elution 5 | 9.40 |
| Elution 6 | 10.07 |

### Example 3: RNA Capture with Acidic Elution

### Sample Preparation

Escherichia coli cells were grown in LB media (American Bioinnovations), then spun down and resuspended in 3 mL of 1× Phosphate Buffered Saline (Sigma-Aldrich). This was then pipetted into Tempus Blood RNA tubes (Thermo Fisher) containing 6 mL stabilizing solution comprising GnSCN. Triton X-100 (Sigma-Aldrich) was added to the solution for a final concentration of 1%. The prepared sample was mixed by inversion and stored for nine days at 4° C. Immediately prior to RNA capture, the sample was stored for 30 minutes at room temperature.

### RNA Capture

1 mL of the prepared sample was flowed through the Oligo Affinity Support Column (˜60 μl volume) containing the LNA capture probe SEQ ID NO:2 for hybridization. Immediately following hybridization, 1 mL of Wash Buffer 1 (3 M guanidinium thiocyanate (Sigma-Aldrich), 10 mM Tris-HCl (pH 8.5, Rockland), 1% Triton X-100 (Sigma-Aldrich)) was flowed through the column, followed by 2 mL of Wash Buffer 2 (150 mM NaCl (Sigma-Aldrich), 100 mM Tris-HCl (pH 8.5, Rockland), 0.02% Triton X-100 (Sigma-Aldrich)), followed by 4 mL of Wash Buffer 3 (150 mM NaCl (Sigma-Aldrich), 5 mM Tris-HCl (pH 8.5, Rockland)). After Wash Buffers 1, 2, and 3, the column was cleared by air.

400 μl of elution solution (0.1 M HCl (Merck)) was flowed through the column and collected in 10 fractions of 40 μl each. The fractions were neutralized with 0.5 M NaOH (Alfa Aesar). Tris-HCl (pH 8.0, Thermo Fisher) was added to a final concentration of 50 mM. The elutions were stored at −80° C. for one day prior to quality control testing.

### Quantitative Reverse Transcription PCR

The first 6 fractions of elution solution were reverse transcribed and amplified using specific primers and a probe for conserved bacterial 16S. qRT-PCR reactions (25 μl) were prepared by mixing 1× HawkZ05 Fast One-Step RT-PCR Master (Roche), 1.5 mM manganese acetate (Roche), 1 mM MgCl2 (Thermo Fisher), 4% DMSO (Thermo Fisher), 0.4 μM forward primer (IDT), 0.4 μM reverse primer (IDT), 0.2 μM probe (IDT) and eluted RNA as template (5 μl). The cycling protocol was performed as following: 5 minutes at 55° C., 10 minutes at 60° C., 15 minutes at 65° C., 3 minutes at 94° C., and 60 repetitions of the following cycle: 10 seconds at 94° C., 15 seconds at 60° C., and 3 seconds at 72° C. (plate fluorescence readings captured at the 72° C. step of each cycle). The specific oligonucleotides used were as described in Table 2.

### Results and Discussion

Escherichia coli was grown and suspended in Phosphate Buffered Saline then added to Tempus RNA tubes and combined with additional detergent. This solution was subject to the LNA 16S Oligo Affinity Support Column capture protocol as above. The quality of the captured RNA was subsequently assessed by qRT-PCR.

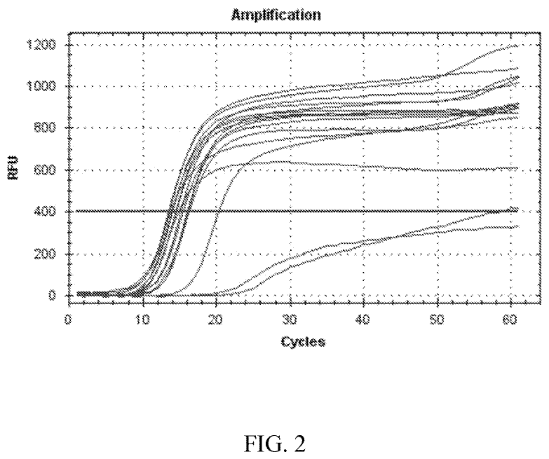

FIG. 2 and Table 4 show the results of qRT-PCR, in which 6 elutions were amplified in qRT-PCR reactions using specific primers and a probe for conserved bacterial 16S rRNA. All elutions show considerable amplification, with the most 16S rRNA detected in Elution 3. The results demonstrate the ability to selectively capture and elute amplifiable bacterial RNA in acidic buffer (pH<4).

TABLE 4
qRT-PCR average cycle threshold.

| Sample | Average Cycle Threshold |
|---|---|
| Negative Control | 58.53 |
| Positive Control | 14.32 |
| Elution 1 | 16.15 |
| Elution 2 | 13.83 |
| Elution 3 | 13.71 |
| Elution 4 | 14.51 |
| Elution 5 | 15.31 |
| Elution 6 | 18.22 |

### Example 4: Capture of mRNA

### Sample Preparation

3 mL of human blood was drawn into Tempus Blood RNA tubes (Thermo Fisher) containing 6 mL stabilizing solution comprising GnSCN. Triton X-100 (Sigma-Aldrich) was added to the solution for a final concentration of 1%. The prepared sample was mixed by inversion and stored for 30 minutes at room temperature prior to RNA capture.

### RNA Capture

2 mL of the prepared sample was flowed through the Oligo Affinity Support Column (˜60 μl volume) containing the LNA capture probe SEQ ID NO:1 for hybridization. Immediately following hybridization, 1 mL of Wash Buffer 1 (3 M guanidinium thiocyanate (Sigma-Aldrich), 10 mM Tris-HCl (pH 8.5, Rockland), 1% Triton X-100 (Sigma-Aldrich)) was flowed through the column, followed by 2 mL of Wash Buffer 2 (150 mM NaCl (Sigma-Aldrich), 100 mM Tris-HCl (pH 8.5, Rockland), 0.02% Triton X-100 (Sigma-Aldrich)), followed by 4 mL of Wash Buffer 3 (150 mM NaCl (Sigma-Aldrich), 5 mM Tris-HCl (pH 8.5, Rockland)). After Wash Buffers 1, 2, and 3, the column was cleared by air.

400 μl of elution solution (0.1 M NaOH (Alfa Aesar)) was flowed through the column and collected in 10 fractions of 40 μl each. The fractions were neutralized with 0.5 M HCl (Merck). Tris-HCl (pH 8.0, Thermo Fisher) was added to a final concentration of 50 mM. The elutions were stored at −80° C. for two days prior to quality control testing.

### Quantitative Reverse Transcription PCR

The first 6 fractions of elution solution were reverse transcribed and amplified using specific primers and a probe for GAPDH, a human housekeeping gene where mRNA transcripts would be expected to be present in all human samples. qRT-PCR reactions (25 μl) were prepared by mixing 1× HawkZ05 Fast One-Step RT-PCR Master (Roche), 1.5 mM manganese acetate (Roche), 1 mM MgCl2 (Thermo Fisher), 4% DMSO (Thermo Fisher), 0.5 μM forward primer (IDT), 0.5 μM reverse primer (IDT), 0.25 μM probe (IDT) and eluted RNA as template (5 μL). The cycling protocol was performed as following: 5 minutes at 55° C., 10 minutes at 60° C., 15 minutes at 65° C., 3 minutes at 94° C., and 60 repetitions of the following cycle: 10 seconds at 94° C., 15 seconds at 56° C., and 3 seconds at 72° C. (plate readings captured at the 72° C. step of each cycle). The specific oligonucleotides used were as follows (Table 5):

TABLE 5
Primer and probe sequences.

| Function | Sequence |
|---|---|
| forward | 5′ AGGGTGGTGGACCTCAT 3′ (SEQ ID NO: 6) |
| primer | |
| reverse | 5′ TGAGTGTGGCAGGGACT 3′ (SEQ ID NO: 7) |
| primer | |
| qPCR | 5′-/56- |
| probe | FAM/CAGCAAGAG/ZEN/CACAAGAGGAAGAGAGA/ |
| | 3IABKFQ/-3′ (SEQ ID NO: 8) |

### Results and Discussion

Human blood was drawn into Tempus RNA tubes and combined with additional detergent. This solution was subject to the LNA Poly-T Oligo Affinity Support Column capture protocol as above. The quality of the captured RNA was subsequently assessed by qRT-PCR.

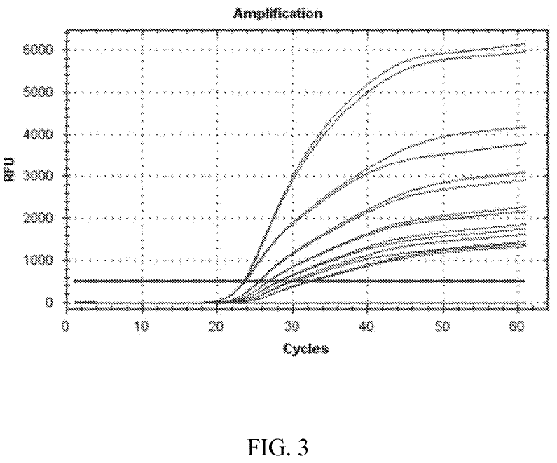

FIG. 3 and Table 6 show the results of qRT-PCR, in which 6 elutions were amplified in qRT-PCR reactions using specific primers and a probe for GAPDH. All elutions show considerable amplification, with the most human RNA detected in Elution 1. The results demonstrate the ability to selectively capture and elute amplifiable human RNA in Alkaline buffer (pH>12) without substantial loss of full-length RNA as would be expected under alkaline conditions.

TABLE 6
qRT-PCR average cycle threshold.

| Sample | Average Cycle Threshold |
|---|---|
| Negative Control | N/A |
| Positive Control | 23.47 |
| Elution 1 | 23.65 |
| Elution 2 | 25.46 |
| Elution 3 | 26.73 |
| Elution 4 | 28.13 |
| Elution 5 | 28.78 |
| Elution 6 | 32.19 |

### Example 5: DNA Capture

### Sample Preparation

Escherichia coli cells were grown in LB media (American Bioinnovations), then spun down and resuspended in 3 mL of 1× Phosphate Buffered Saline (Sigma-Aldrich). This was then pipetted into Tempus Blood RNA tubes (Thermo Fisher) containing 6 mL stabilizing solution comprising GnSCN. Triton X-100 (Sigma-Aldrich) was added to the solution for a final concentration of 1%. The prepared sample was mixed by inversion and stored for 30 minutes at room temperature prior to the capture experiment.

Nucleic acid Capture

1 mL of the prepared sample was flowed through the Oligo Affinity Support Column (˜60 μl volume) containing the LNA capture probe SEQ ID NO:2 for hybridization. Immediately following hybridization, 1 mL of Wash Buffer 1 (3 M guanidinium thiocyanate (Sigma-Aldrich), 10 mM Tris-HCl (pH 8.5, Rockland), 1% Triton X-100 (Sigma-Aldrich)) was flowed through the column, followed by 1 mL of Wash Buffer 2 (150 mM NaCl (Sigma-Aldrich), 100 mM Tris-HCl (pH 8.5, Rockland), 0.02% Triton X-100 (Sigma-Aldrich)), followed by 1 mL of Wash Buffer 3 (150 mM NaCl (Sigma-Aldrich), 5 mM Tris-HCl (pH 8.5, Rockland)). After Wash Buffers 1, 2, and 3, the column was cleared by air.

400 μl of elution solution (0.1 M NaOH (Alfa Aesar)) was flowed through the column and collected in 10 fractions of 40 μl each. The fractions were neutralized with 0.5 M HCl (Merck). Tris-HCl (pH 8.0, Thermo Fisher) was added to a final concentration of 50 mM. The elutions were stored at −80° C. for seven days prior to quality control testing.

### Quantitative PCR

The first 6 fractions of elution solution were amplified using specific primers and a probe for conserved bacterial 16S. qPCR reactions (20 μl) were prepared by mixing 1× ThermoPol Buffer (NEB), 0.2 mM dNTPs (Roche), 0.4 μM forward primer (IDT), 0.4 μM reverse primer (IDT), 0.2 μM probe (IDT), 1 U Taq DNA Polymerase (NEB) and eluted DNA as template (5 μl). The cycling protocol was performed as following: 2 minutes at 50° C., 10 minutes at 95° C. and 60 repetitions of the following cycle: 15 seconds at 95° C. and 15 seconds at 60° C. (plate fluorescence readings were captured at the 60° C. step of each cycle). The specific oligonucleotides used were as follows (Table 7):

TABLE 7
Primer and probe sequences.

| Function | Sequence |
|---|---|
| forward | 5′ GAT GGT GCA GGT GGT GCA /iSuper-dT/GG 3′ (SEQ ID NO: 9) |
| primer | |
| reverse | 5′ GTG GGT TGC GCT CGT TGC GG 3′ (SEQ ID NO: 10) |
| primer | |
| qPCR probe | 5′ /56-FAM/G+TCG+TC+AGC+TCG+TG/Iowa Black FQ/ 3′ (SEQ ID |
| | NO: 11) |

### Results and Discussion

Escherichia coli was grown and suspended in Phosphate Buffered Saline then added to Tempus RNA tubes and combined with additional detergent. This solution was subject to the LNA 16S Oligo Affinity Support Column capture protocol as above. The quality of the captured DNA was subsequently assessed by qPCR.

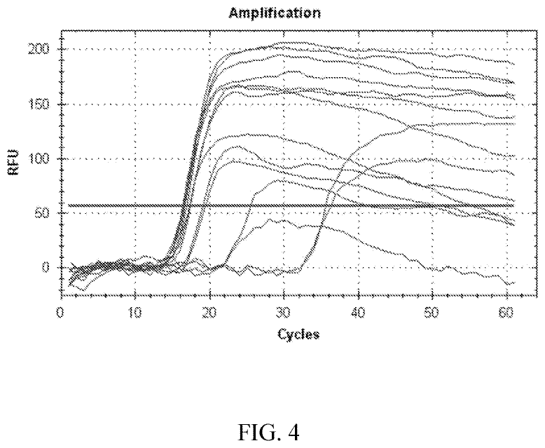

FIG. 4 and Table 8 show the results of qPCR, in which 6 elutions were amplified in qPCR reactions using specific primers and a probe for conserved bacterial 16S DNA.

TABLE 8
qRT-PCR average cycle threshold.

| | Average Cycle |
|---|---|
| Sample | Threshold |
| Negative Control | 35.94 |
| Elution 1 | 25.52 |
| Elution 2 | 19.23 |
| Elution 3 | 17.33 |
| Elution 4 | 16.71 |
| Elution 5 | 16.96 |
| Elution 6 | 16.37 |

### Example 6: Capture and Elution of Bacterial 16S rRNA and DNA from Human Blood

### Sample Preparation

3 mL of human blood was drawn into Tempus Blood RNA tubes (Thermo Fisher) containing 6 mL stabilizing solution comprising GnSCN. 10 CFU/μl stock solution of Klebsiella pneumoniae (BEI Resources) was prepared in Tempus tubes and 300 μl of it was added to the Tempus Blood RNA tube containing human blood to prepare 1000 CFU/mL blood in Tempus Blood RNA tube. Triton X-100 (Sigma-Aldrich) was added to the solution for a final concentration of 1%. The prepared sample was mixed by inversion and stored at 4° C. Immediately prior to RNA capture, the sample was stored for 30 minutes at room temperature.

### RNA Capture

1 mL of the prepared sample was flowed through the Oligo Affinity Support Column (˜60 μl volume) containing the LNA capture probe SEQ ID NO:2 for hybridization. Immediately following hybridization, 1 mL of Wash Buffer 1 (3 M guanidinium thiocyanate (Sigma-Aldrich), 10 mM Tris-HCl (pH 8.5, Rockland), 1% Triton X-100 (Sigma-Aldrich)) was flowed through the column, followed by 2 mL of Wash Buffer 2 (150 mM NaCl (Sigma-Aldrich), 100 mM Tris-HCl (pH 8.5, Rockland), 0.02% Triton X-100 (Sigma-Aldrich)), followed by 4 mL of Wash Buffer 3 (150 mM NaCl (Sigma-Aldrich), 5 mM Tris-HCl (pH 8.5, Rockland)). After Wash Buffers 1, 2, and 3, the column was cleared by air.

400 μl of elution solution (0.1 M NaOH (Alfa Aesar)) was flowed through the column and collected in 10 fractions of 40 μl each. The fractions were neutralized with 0.5 M HCl (Merck). Tris-HCl (pH 8.0, Thermo Fisher) was added to a final concentration of 50 mM. The elutions were stored at −80° C. prior to testing.

### Quantitative Reverse Transcription PCR

The first 6 fractions of elution solution were reverse transcribed and amplified using specific primers and a probe for conserved bacterial 16S. qRT-PCR reactions (25 μl) were prepared by mixing 1× HawkZ05 Fast One-Step RT-PCR Master (Roche), 1.5 mM manganese acetate (Roche), 1 mM MgCl2 (Thermo Fisher), 4% DMSO (Thermo Fisher), 0.4 μM forward primer (IDT), 0.4 μM reverse primer (IDT), 0.2 μM probe (IDT) and eluted RNA as template (5 μl). The cycling protocol was performed as following: 5 minutes at 55° C., 10 minutes at 60° C., 15 minutes at 65° C., 3 minutes at 94° C., and 60 repetitions of the following cycle: 10 seconds at 94° C., 15 seconds at 60° C., and 3 seconds at 72° C. (plate fluorescence readings were captured at the 72° C. step of each cycle). The specific oligonucleotides used were as in Table 7.

### Quantitative PCR

The first 6 fractions of elution solution were amplified using specific primers and a probe for conserved bacterial 16S. qPCR reactions (20 μl) were prepared by mixing 1× ThermoPol Buffer (NEB), 0.2 mM dNTPs (Roche), 0.4 μM forward primer (IDT), 0.4 μM reverse primer (IDT), 0.2 μM probe (IDT), 1 U Taq DNA Polymerase (NEB) and eluted DNA as template (5 μl). The cycling protocol was performed as following: 2 minutes at 50° C., 10 minutes at 95° C. and 60 repetitions of the following cycle: 15 seconds at 95° C. and 15 seconds at 60° C. (plate fluorescence readings were captured at the 60° C. step of each cycle). The specific oligonucleotides used were the same as used for qRT-PCR (Table 7).

### Results and Discussion

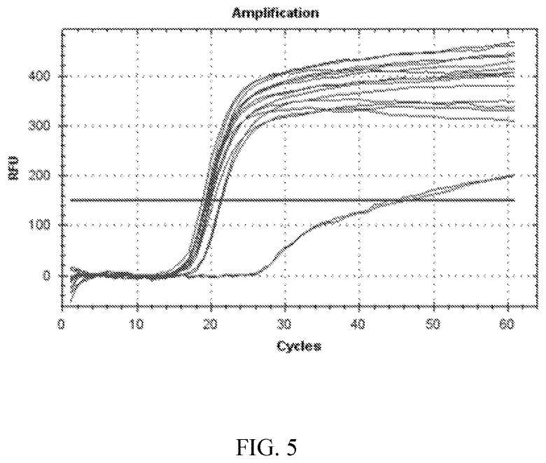

Klebsiella pneumoniae was spiked into human blood stored in Tempus RNA tubes at a concentration of 1000 CFU/mL blood and combined with additional detergent. This solution was subject to the LNA 16S Oligo Affinity Support Column capture protocol as above. The quality of the captured RNA and DNA was subsequently assessed by qRT-PCR and qPCR respectively. FIG. 5 and Table 9 show the results of qRT-PCR, in which 6 elutions were amplified in qRT-PCR reactions using specific primers and a probe for conserved bacterial 16S rRNA. All elutions show considerable amplification, with the most 16S rRNA detected in Elution 4.

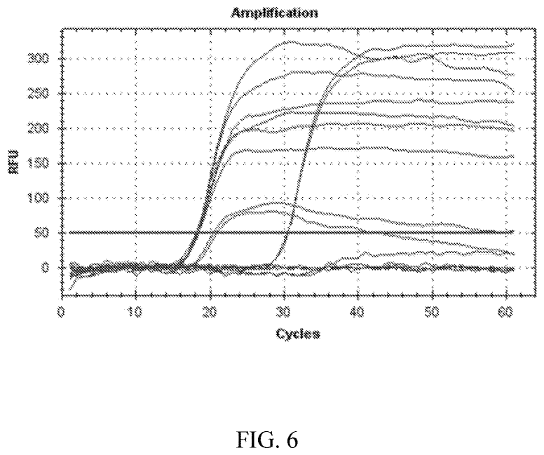

FIG. 6 and Table 9 show the results of qPCR, in which 6 elutions were amplified in qPCR reactions using specific primers and a probe for conserved bacterial 16S rRNA. There was no DNA amplification detected in the first two elutions, but was detected in later elutions.

TABLE 9
Primer and probe sequences.

| | Average Cycle | Average Cycle |
|---|---|---|
| Sample | Threshold RNA | Threshold DNA |
| Negative Control | 45.75 | N/A |
| Positive Control | 19.06 | 30.44 |
| Elution 1 | 21.33 | N/A |
| Elution 2 | 20.16 | N/A |
| Elution 3 | 19.44 | 20.64 |
| Elution 4 | 19.37 | 18.23 |
| Elution 5 | 19.79 | 18.37 |
| Elution 6 | 19.81 | 18.15 |

What is claimed is:

1. A method of enriching a target nucleic acid in a sample, the method comprising:
(a) contacting the sample with a capture probe under a condition that allows for formation of a complex comprising the capture probe and the target nucleic acid,
wherein the capture probe comprises one or more nucleotide analogs; and
(b) eluting the target nucleic acid from the capture probe, using an elution buffer at a pH sufficient to disrupt the interaction between the target nucleic acid and the capture probe,
wherein the target nucleic acid is enriched.

2. The method of claim 1, wherein the capture probe comprising nucleotide analogs has increased binding affinity to the target nucleic acid, as compared to a capture probe without one or more of the nucleotide analogs.

3. The method of claim 1 or 2, wherein at least about 40% of the nucleotides of the capture probe are nucleotide analogs.

4. The method of any one of claims 1-3, wherein the one or more nucleotide analogs are selected from any of a locked nucleic acid (LNA), peptide nucleic acid (PNA), a xeno nucleic acid (XNA), a glycol nucleic acid (GNA), a threose nucleic acid (TNA), a morpholino, a bridged nucleic acid (BNA), an O-methyl substituted RNA, a nucleotide with a modified sugar, base group, or backbone, or any combination thereof.

5. The method of any one of claims 1-4, wherein the capture probe is at least about 70% complementary to at least a portion of the target nucleic acid.

6. The method of claim 5, wherein each of the one or more nucleotide analogs is a LNA.

7. The method of any one of claims 1-6, wherein the elution buffer has a pH of about 11 or greater.

8. The method of any one of claims 1-6, wherein the elution buffer has a pH of about 3 or less.

9. The method of any one of claims 1-8, wherein the target nucleic acid is a RNA.

10. The method of any one of claims 1-8, wherein the target nucleic acid is a DNA.

11. The method of any one of claims 1-10, wherein the capture probe is attached to a solid surface.

12. The method of any one of claims 1-11, wherein the capture probe is covalently attached to a solid surface.

13. The method of any one of claims 1-12, further comprising neutralizing the pH of the elution buffer after eluting the target nucleic acid.

14. The method of any one of claims 1-13, further comprising obtaining the sample.

15. The method of claim 14, wherein the sample comprises a cell or cellular material.

16. The method of claim 15, further comprising subjecting the sample to a lysing buffer comprising a chaotropic agent prior to the enrichment step.

17. The method of any one of claims 1-16, further comprising detecting if the target nucleic acid is present.

18. The method of claim 17, wherein the detecting the presence of the target nucleic acid is performed after eluting the target nucleic acid from the capture probe.

19. A method of detecting a target nucleic acid in a sample, the method comprising:
(a) contacting the sample with a capture probe under a condition that allows for formation of a complex comprising the capture probe and the target nucleic acid,
wherein the capture probe comprises one or more LNA nucleotides;
(b) eluting the target nucleic acid from the capture probe using an elution buffer at a pH sufficient to disrupt the interaction between the target nucleic acid and the capture probe;
(c) neutralizing the pH of the elution buffer after the eluting step; and
(d) detecting the target nucleic acid.

20. The method of claim 19, wherein the detecting step comprises amplification using a DNA or RNA polymerase enzyme and subsequent detection of products of the amplification reaction.
